(19) United States
(12) Patent Application Publication
(10) Pub. No.: US 2008/0294103 A1
(43) Pub. Date: Nov. 27, 2008

(54) Constructive disposition applied to balloon catheters

(76) Inventor: Adamastor Humberto Pereira, Porto Alegre (BR)

Correspondence Address: PATENT LAW AGENCY, LLC, 3146 North Verdugo Road, Glendale, CA, 91208-1665, US

(21) Appl. No.: 12/220,391

(22) Filed: Jul. 24, 2008

Related U.S. Application Data
(63) Continuation of application No. PCT/BR06/00088, filed on May 4, 2006.

(30) Foreign Application Priority Data
Feb. 24, 2006 (BR) MU8600250-3

(51) Int. Cl. A61M 25/10

(52) U.S. Cl. 604/103.1

(57) ABSTRACT

Balloon catheter consisting of a multilumen shaft (1) which contains, at least, two internal channels (8) and (9); a multilumen shaft (1) this ending at its proximal end by a Y connection (4) were the internal channels (8) and (9) get separated until the Luer Lock connections (5); multilumen shaft (1) still ended at its distal and by the body of the balloon (2), which is crossed by the simple shaft (7) containing the application of at least two radio-opaque markers (3) presenting conical geometry, having a cylindrical portion at its proximal end (6) and manufactured in such a way that it may present or not a predetermined curvature.

### CROSS-REFERENCE TO RELATED APPLICATIONS

This Application is a CONTINUATION of a co-pending International Patent Application No. PCT/BR2006/000088 with an international filing date of 4 May 2006 that designated the United States of America, which claims the benefit of priority of the Federative Republic of Brazil Patent Application No. MU8600250-3, with a filing date of 13 Jul. 2006, the entire disclosures of all Applications is expressly incorporated by reference in their entirety herein.

### BACKGROUND OF THE INVENTION

1. Field of the Invention

This utility model patent aims at a balloon catheter model, to be used in surgical medical procedures, to which an original construction was given, so as to improve and extend its use and efficiency in relation to the similar ones.

2. Description of Related Art

Balloon catheters are devices used in surgical medical procedures with the most varied purposes, including dilatation of vascular narrowing, temporary tamponing of hemorrhages, application of stents, among others. Typically, they are differentiated by their external design, calibers and lengths, manufacturing materials and expansion radial force. They may be basically of three types: complacent—balloon catheter manufactured in elastic material that accompanies the anatomy or local shape; non-complacent—balloon catheter manufactured in non-elastic material that assumes a pre-determined shape in its project; or semi-complacent—balloon catheter manufactured with material having limited elastic characteristics.

In general, a balloon catheter is made up of various parts, including: multilumen shaft, balloon body; radio-opaque marking; Luer Lock connection; and Y connection.

The balloon catheters in use today have been developed to be inserted when they are empty up to the place of use, where they are inflated and subsequently emptied, so as to remove them. According to this functioning principle, they do not present good capacity or even indication for intraluminal navigation at inflated stage.

For this reason, the drawings of balloon catheters currently available do not allow its use in endoprosthesis delivery systems, for example, which cause their construction and design limitations to be subject to constant evolution and technical improvement.

Accordingly, in light of the current state of the art and the drawbacks to current rod and beverage container holders mentioned above, a need exists for an apparatus for carrying objects, non-limiting examples of which may include elongated bars or fishing rods, and for holding of objects including rods and or beverage containers such as bottles or cups within the same unit. In addition, a need exists for such an apparatus that would detachable mount and removably secure onto a permanent structures at any orientation using a novel universal mount joint.

### BRIEF SUMMARY OF THE INVENTION

In view of the problems presented and in order to overcome them, a new device was developed in terms of balloon catheter, which is the object of an innovative shape, aiming at providing it with intraluminal navigation capacity when inflated. This capacity is added to the others normally expected for a balloon catheter, without jeopardizing its performance in situations already usual.

The innovative shape employed in the construction of the balloon body is of conic geometry, characterized for having a cylindrical segment at its proximal portion, which allows the use of the balloon catheter on a regular manner and also the fixing or anchoring of surgical medical accessories that need to be intraluminarly inserted in the patient.

Besides, this innovative shape may present or not predetermined curves that makes it easy the negotiation of the balloon catheter through anatomic twisted parts during its passage throughout the same.

This construction form of the balloon catheter solves the inconveniences raised and makes the device even more versatile as a tool for surgical medical use.

The attached drawings show the disposition in balloon catheter, object of this patent.

These and other features, aspects, and advantages of the invention will be apparent to those skilled in the art from the following detailed description of preferred non-limiting exemplary embodiments, taken together with the drawings and the claims that follow.

### BRIEF DESCRIPTION OF THE DRAWINGS

It is to be understood that the drawings are to be used for the purposes of exemplary illustration only and not as a definition of the limits of the invention. Throughout the disclosure, the word “exemplary” is used exclusively to mean “serving as an example, instance, or illustration.” Any embodiment described as “exemplary” is not necessarily to be construed as preferred or advantageous over other embodiments.

Referring to the drawings in which like reference character(s) present corresponding part(s) throughout:

FIG. 1 shows it in a plan representation with its various parts and the inter-relationship thereof;

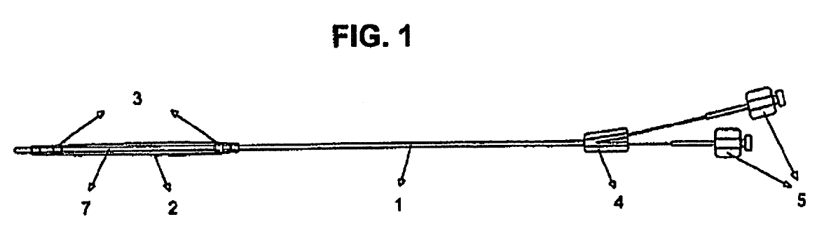

FIG. 2 shows it in details at the distal portion;

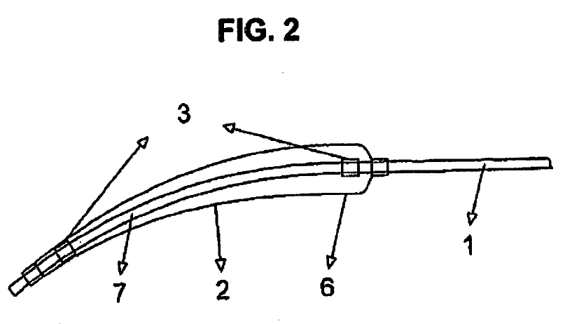

FIG. 3 shows in cut at the medium portion.

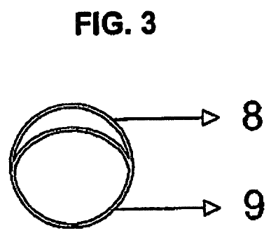

### DETAILED DESCRIPTION OF THE INVENTION

The detailed description set forth below in connection with the appended drawings is intended as a description of presently preferred embodiments of the invention and is not intended to represent the only forms in which the present invention may be constructed and or utilized.

According to the illustrations in the above-mentioned figures, the balloon catheter model, object of this patent, consists in a multilumen shaft (1) which contains, at least, two independent and isolated internal channels, the first being called balloon channel (8) used to inflate and deinflate the body of the balloon (2) and the second called guiding channel (9) used for passage of the guiding thread; multilumen shaft (1) this ends at its proximal end by Y connection (4) where the internal channels (8) and (9) separate in independent tubes up to their respective Luer Lock connections (5); multilumen shaft (1) still ended at its distal end by the body of the balloon (2), which is crossed by the simple tube (7) this being of single channel and having the application of at least two radio-opaque markers (3) located at its distal and proximal ends and have the function of making reference, in the monitor of a fluoroscopy equipment, to the exact dimension an position of the balloon body (2) in the patient's body.

In this disposition, the balloon body (2) is of conic geometry, presenting a cylindrical portion at its proximal end (6) and being also manufactured in such a way that it may present or not a pre-determined curvature.

The conic shape applied to the balloon body (2), preferably manufactured with non-complacent material, aims at a new concept of use, whereby the balloon catheter is inserted on intraluminar manner already inflated in the patient. This conic shape provides an intraluminal navigation capacity without the use of ancillary accessories, in addition to a guiding thread and ensures reliability, comfort and easiness of handling, both to the patient and the medical team.

The pre-determined curvature of the balloon body (2) aims at making it easy the navigation of the balloon catheter throughout anatomic twisted parts. Now, the cylindrical portion (6) at the proximal portion of the balloon catheter (2) aims at its utilization as an anchorage or fixing point for various accessories, which need to be inserted on a intraluminar manner, such as: stent inserting systems, diagnosis catheters, etc., and It may also be used as a dilation zone for narrowing as occurring with balloon catheters already existing in the market.

Logically, the balloon catheter with such construction may be obtained in several calibers, diameters, lengths and curvatures in order to meet different needs of users of this type of device.

Although the invention has been described in considerable detail in language specific to structural features and or method acts, it is to be understood that the invention defined in the appended claims is not necessarily limited to the specific features or acts described. Rather, the specific features and acts are disclosed as preferred forms of implementing the claimed invention. Stated otherwise, it is to be understood that the phraseology and terminology employed herein, as well as the abstract, are for the purpose of description and should not be regarded as limiting.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, proximal, distal, parallel, perpendicular, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) is not used to show a serial or numerical limitation but instead is used to distinguish or identify the various members of the group.

In addition, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of,” “act of,” “operation of,” or “operational act of” in the claims herein is not intended to invoke the provisions of 35 U.S.C. 112, Paragraph 6.

What is claimed is:

1. Constructive disposition applied to balloon catheters formed by a multilumen shaft (1) which contains, at least, two independent and isolated internal channels, the first being called balloon channel used to inflate and deinflate the body of the balloon and the second being called guiding channel used for passage of the guiding thread; multilumen shaft this ends at its proximal end by Y connection where the internal channels and separate in independent tubes up to their respective Luer Lock connections; multilumen shaft still ended at its distal end by the body of the balloon, which is crossed by the simple shaft this being of single channel and having the application of at least two radio-opaque markers located at its distal and proximal ends and have the function of making reference, in the monitor of a fluoroscopy equipment, to the exact dimension an position of the balloon body whenever the balloon catheter is inside the patient's body characterized by balloon body presenting a conical geometry, which has a cylindrical portion at it proximal end and is preferably manufactured, with non complacent material and in such a way it may present or not a pre-determined curvature.
